(19) United States
(12) Patent Application Publication
(10) Pub. No.: US 2006/0067949 A1
(43) Pub. Date: Mar. 30, 2006

(54) Immunization with porphyromonas gingivalis protects against heart disease

(76) Inventors: Caroline A Genco, Brookline, MA (US); Frank C Gibson, Jamaica, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/535,490

(22) PCT Filed: Nov. 18, 2003
(86) PCT No.: PCT/US03/37013

§ 371 Date: May 17, 2005

Related U.S. Application Data
(60) Provisional application No. 60/427,318, filed on Nov. 18, 2002.

(51) Int. Cl. A61K 39/02

(52) U.S. Cl. 424/234.1

(57) ABSTRACT

The invention is directed to a method of preventing and treating a patient having a risk factor and/or a symptom of cardiovascular disease using an immunogenic composition comprising an immunogenically effective portion of Porphyromonas gingivalis in a pharmaceutically effective carrier substance, and a vaccine for same.

### CROSS REFERENCE TO RELATED APPLICATIONS

This application claims priority from U.S. Provisional Application No. 60/427,318, filed on Nov. 18, 2002, entitled IMMUNIZATION WITH PORPHYROMONAS GINGIVALIS PROTECTS AGAINST HEART DISEASE, the whole of which is hereby incorporated by reference herein.

### STATEMENT REGARDING FEDERALLY SPONSORED RESEARCH OR DEVELOPMENT

Part of the work leading to this invention was carried out with United States Government support provided under grants from the National Institutes of Health, Grant Nos. DE 13191 and DE 12517. Therefore, the U.S. Government has certain rights in this invention.

### BACKGROUND OF THE INVENTION

Cardiovascular disease (CVD) is one of the primary causes of death in the western world (Ross 1993), and despite identification and thorough study of several important factors that predispose humans to CVD including diet, metabolism, exercise, and genetics, an incomplete picture of the etiology of CVD is evident (Libby 2001). A more thorough understanding of the mechanisms underlying the pathogenesis of CVD has emerged due in part to the use of gene-targeted animals such as the ApoE^{-/-} knockout (ApoE^{-/-}) mouse that develops severe hyperlipidemia, and accelerated atheroma formation (Zhang et al., 1992; Plump et al., 1992). Inflammation is a determining factor for development of CVD, with emphasis on a mononuclear cellular infiltrate (Mach et al., 1999), the host response to ox-LDL (Steinberg 1997), cytokines (Gupta et al., 1997), C-reactive protein levels (Liuzzo et al., 1994), cell adhesion molecule expression (Cybulsky et al., 2001), and identification of Toll-like receptor (TLR)-4 at the site of atherosclerotic plaque accumulation (Xu et al., 2001). TLRs are an emerging group of pattern recognition molecules that mediate the innate host response to microbes (Lien et al., 2002) and selectively up-regulated following infection (Shuto et al., 2002; Wang et al., 2000). Chronic infectious diseases have been suggested as being associated with CVD (Hansson et al., 2002; Epstein et al., 1999; Mach et al., 2002; Beck et al., 2001; Wu et al., 2000). However, other studies show contradictory results (Wright et al., 2000; Hujoel et al., 2000; Beck et al., 1998). Identifying the causal agent of CVD and providing an appropriate vaccination protocol that would not only treat but also act as a prophylactic in CVD would be desirable.

### BRIEF SUMMARY OF THE INVENTION

The invention is directed to a method of using an immunogenically effective portion of Porphyromonas gingivalis for the prevention and/or treatment of cardiovascular disease. The method of the invention comprises administering to a patient having a symptom of cardiovascular disease an immunogenic composition comprising an immunogenically effective portion of Porphyromonas gingivalis in a pharmaceutically effective carrier substance. The method also provides administering the same to a patient who is at risk of developing cardiovascular disease.

The classic symptoms of cardiovascular disease include, but are not limited to, increased levels of a cytokine or other protein indicative of inflammation, atherosclerosis or an atheroma formation in the aortic arch. Various risk factors for cardiovascular disease include, but are not limited to, smoking, hypertension or high blood pressure, blood cholesterol levels (>200 mg/dl total cholesterol or 160 for LDL), diabetes, obesity, lack of exercise, family history of heart disease, stress, oral contraceptives and excessive alcohol consumption.

Also included within the invention is a vaccine effective as a prophylactic and/or therapeutic treatment against cardiovascular disease comprising a therapeutically effective quantity of an immunogenically effective portion of heat-killed Porphyromonas gingivalis.

### BRIEF DESCRIPTION OF THE FIGURES

Other features and advantages of the invention will be apparent from the following description of the preferred embodiments thereof and from the claims, taken in conjunction with the accompanying drawings, in which:

FIG. 1A is a graph showing serum analysis (n=10 mice/group) of total cholesterol and triglyceride levels from unchallenged (black bars), wild type P. gingivalis (gray bars) and the P. gingivalis FimA- mutant (diagonally hatched bars) challenged mice;

FIG. 1B is a graph showing serum levels of P. gingivalis-specific IgG prior to oral challenge (blue bars) and at 6-weeks following challenge (purple bars) with wild type P. gingivalis (WT) or the FimA- mutant (FimA-);

FIG. 1C is a photograph showing atherosclerotic plaque present on the intimal surface of the aortic arches of unchallenged mice (magnification 40×, magnification bar=1 mm);

FIG. 1D is a photograph showing atherosclerotic plaque present on the intimal surface of the aortic arches of wild type P. gingivalis mice (magnification 40×, magnification bar=1 mm);

FIG. 1E is a photograph showing atherosclerotic plaque present on the intimal surface of the aortic arches of FimA- mutant mice (magnification 40×, magnification bar=1 mm);

FIG. 1F is a bar graph showing morphometric analysis of the total area of atherosclerotic plaque of unchallenged (None), wild type P. gingivalis (WT) or FimA- mutant challenged mice (FimA-) (*=P<0.05; NS=not significant vs. unchallenged mice);

FIG. 2A is a RT-PCR amplification of TLR-2 and TLR-4 mRNA from aortic arch tissue of unchallenged (None), wild type P. gingivalis (WT) and mutant P. gingivalis (FimA-) challenged mice;

FIG. 2B is an immunohistochemical confirmation of TLR-2 and TLR-4 expression in aortic tissue of mice (irrelevant isotype-matched antibody (IRR-Ab) (magnification 200×, magnification bar=10 μm);

FIG. 3A is a FACS analysis of TLR-2 and TLR-4 expression on HAEC cultured with wild type P. gingivalis (red trace) or the FimA- mutant (blue trace) at a MOI of 100 and unstimulated cells functioned as controls (shaded black trace) (as a positive control stimulus for TLR-2 and TLR-4 expression, cells were stimulated with wild type P. gingivalis at a MOI of 100 (shaded black trace));

FIG. 3B is a FACS analysis of HAEC cultured with a high dose (10 μg/ml; red trace) or low dose (1 μg/ml; blue trace) of purified P. gingivalis FimA protein or unstiumulated cells (black trace) (as a positive control stimulus for TLR-2 and TLR-4 expression, cells were stimulated with wild type P. gingivalis at a MOI of 100 (shaded black trace));

FIG. 4A is a photograph of the atherosclerotic plaque on the intimal surface of the aortic arch of unchallenged ApoE^{-/-} mice after 6-weeks (magnification 40×, magnification bar=1 mm);

FIG. 4B is a photograph of the atherosclerotic plaque on the intimal surface of the aortic arch of ApoE^{-/-} mice, 6-weeks following oral challenge with wild type P. gingivalis (magnification 40×, magnification bar=1 mm);

FIG. 4C is a photograph of the atherosclerotic plaque on the intimal surface of the aortic arch of ApoE^{-/-} mice, 6-weeks following oral challenge with wild type P. gingivalis and immunized with heat-killed P. gingivalis (magnification 40×, magnification bar=1 mm); and

FIG. 4D is a bar graph showing morphometric analysis of the total area of atherosclerotic plaque deposited on the intimal surface of the aortic arch of unchallenged (None), wild type P. gingivalis (WT) challenged, or immunized and WT challenged ApoE^{-/-} mice (*=P<0.05 vs. unchallenged; **=P<0.05 vs. WT challenged animals; NS=not significant vs. unchallenged mice).

### DETAILED DESCRIPTION OF THE INVENTION

The invention is directed to a novel method of using an immunogenic composition comprising an immunogenically effective portion of Porphyromonas gingivalis as a vaccine for the prevention and/or treatment of cardiovascular disease (CVD).

Although CVD is a multifactorial disease, using a combinational approach consisting of a defined genetic mutant of P. gingivalis and a site-specific challenge regimen within an effective animal model (ApoE^{-/-}) for assessment of accelerated atherosclerosis, we have been able to show an experimental link between P. gingivalis oral infection and exacerbation of atherosclerotic plaque accumulation. Furthermore, we have determined that a P. gingivalis vaccine reduces the risk of infection-accelerated CVD in the ApoE^{-/-} model. The ApoE^{-/-} knockout mouse has a defect in lipid metabolism, which causes the subsequent development of atherosclerosis, characterized by the deposition of atheromatous plaques containing cholesterol and lipids on the innermost layer of artery walls. As more than 50% of human patients who develop cardiovascular disease have atherosclerosis, the ApoE^{-/-} mouse model is an effective representative of that patient population.

Through experimentation with P. gingivalis, we have found that oral challenge of ApoE^{-/-} mice, in a manner that elicits periodontal disease, also accelerates atheroma formation. Periodontal disease is a chronic inflammatory disease of the periodontium that leads to erosion of the attachment apparatus and supporting bone for the teeth (Armitage 1996). Furthermore, it is one of the most common chronic infectious diseases of humans (Oliver et al., 1998). P. gingivalis is the primary etiologic agent of adult periodontal disease (Holt et al., 1988; Griffen et al., 1998). P. gingivalis has also been detected in human atheromatous tissue by polymerase chain reaction (Haraszthy et al., 2000), indicating that P. gingivalis gains access to the vasculature and localizes at sites of atheroma development. P. gingivalis possesses a broad array of virulence factors including proteases, LPS, capsular polysaccharide, hemagglutinins, and fimbrae (Holt et al., 2000). In vitro studies demonstrate that the fimbrae of P. gingivalis play a significant role in attachment and invasion of endothelial cells (Deshpande et al., 1998), stimulation of cell adhesion molecule production (Khlgatian et al., 2002), and chemokine expression (Nassar et al., 2002). Furthermore, a P. gingivalis fimbriae-deficient mutant failed to elicit oral bone loss in a rat oral infection model (Malek et al., 1994).

Based on the results described herein, we have determined that the mechanism by which P. gingivalis adheres to and/or invades vascular tissue is critical to the stimulation of accelerated atheroma development. For example, a P. gingivalis FimA- mutant failed to accelerate atherosclerosis despite evidence of bacteremia and localization of the mutant in the aorta.

Toll-like receptors (TLRs) are pattern recognition receptors of cells that sense the external environment. It has been reported that TLR-2 and TLR-4 play a role in the host response to P. gingivalis LPS (Bainbridge et al., 2001), and fimbriae (Hajishengallis et al., 2002). However, the importance of the TLR-mediated response during P. gingivalis mediated periodontal disease are unknown. As described herein, we have determined that during P. gingivalis-accelerated atherosclerosis, the host modulates the expression of TLRs. Furthermore, the upregulation of the innate immune response precedes accelerated atheroma development. The outcome of accelerated atherosclerosis is then critical as the mechanism by which an infectious agent adheres to or invades the host in addition to correlating with TLR expression. The in vitro and in vivo data show that only fully invasive P. gingivalis initiates accelerated plaque accumulation. Merely the localization of P. gingivalis in the aortic tissue, capture of non-invasive P. gingivalis by the spontaneously developing atheromatous plaque, or the presence of P. gingivalis is insufficient to drive accelerated atheroma formation. Vaccination according to the method of the invention prohibits the invasive bacteria from targeting the aorta and prevents the stimulation of an immune response caused by P. gingivalis.

The strength of the epidemiological and clinical associations of infectious agents with atherosclerosis can be increased by the demonstration that specific microbes can initiate and sustain growth in human vascular cells as well as in animal models. The development of animal models of atherosclerosis has made it possible to design controlled studies to examine definitively the issue of causality. Numerous investigators studying mechanisms of atherosclerosis have used a murine apolipoprotein E-deficient (ApoE^{-/-}) mouse. Because of the deficiency of ApoE^{-/-}, these animals have high levels of plasma cholesterol and develop atherosclerosis with a reproducible time course. The marked hypercholesterolemia is primarily due to elevated levels of very low and intermediate density lipoproteins. Atherosclerosis in ApoE^{-/-} mice closely resembles that in humans with respect to histology, progression, and dependence on circulating cholesterol. The ApoE^{-/-} mice are commercially available from the Jackson Laboratories in Bar Harbor Maine and have been exceedingly well characterized as to the extent of lesion formation as a function of both diet and time.

Initial reports have demonstrated that intraperiotoneal infection of P. gingivalis in subcutaneous chambers implanted in heterozygous ApoE^{-/-} mice fed a regular diet increases the mean area and the extent of atherosclerotic lesions relative to the uninfected animal. Intravenous infection of P. gingivalis in heterozygous ApoE^{-/-} mice fed a normal chow diet or a high fat diet has also recently been demonstrated to increase plaque formation as compared to uninfected control animals (50). We have determined that oral infection with invasive P. gingivalis, elicits oral bone loss and accelerates atherosclerosis in ApoE^{-/-} mice (reference). Our results indicate that the increase in atherosclerotic lesions was due to active invasion of P. gingivalis, as the fimA mutant did not accelerate atheroma.

Accordingly, in one aspect, the invention is directed to a method of preventing cardiovascular disease by identifying a patient who is at risk of cardiovascular disease and administering to the patient an immunogenic composition comprising an effective amount of an immunogenically active portion of Porphyromonas gingivalis in a pharmaceutically acceptable carrier substance. The invention also includes, in another aspect, a method of treating a patient who shows a symptom of cardiovascular disease by administering a therapeutically effective amount of an immunogenic composition comprising an effective portion of Porphyromonas gingivalis in a pharmaceutically effective carrier substance.

An appropriate patient population for vaccination in accordance with the invention includes, but is not limited to, those who test positive for increased levels of, e.g., C-reactive protein, soluble ICAM, interleukin-6 or interferon-gamma. It is well known in the art that these and other cytokines or proteins can be indicative of inflammation related to cardiovascular disease. Molecular levels of these proteins can be measured in serum samples by methods well known in the art or through commercially available ELISA detection kits. Atherosclerosis, a predominant symptom in patients with CVD, is a condition in which cholesterol, fat, and fibrous tissue build up in medium and large sized artery walls.

A number of risk factors exist that are indicative of the likelihood of the patient developing CVD. Such factors include, but are not limited to, smoking, hypertension or high blood pressure, blood cholesterol levels (>200 mg/dl total cholesterol or 160 for LDL), diabetes, obesity, lack of exercise, family history of heart disease, stress, oral contraceptives and excessive alcohol consumption. A discussion of such risk factors is at www.hmc.psu.edu/cardiovascular/patient/articles/pe099.htm.

In the present invention, a P. gingivalis vaccine as described herein is useful in preventing and/or treating cardiovascular disease. The vaccine is useful, for example, in immunizing an animal, including a human, against accelerated atherosclerosis caused by inflammation in response to a P. gingivalis infection. The invention includes a method of administering an immunogenic composition comprising an effective portion of P. gingivalis. The immunogenically effective portion of P. gingivalis may include, but is not limited to, P. gingivalis heat-killed whole cells; an immunogenically effective portion of P. gingivalis capsular polysaccharide; an immunogenic composition comprising at least one peptide corresponding in sequence to the amino terminus of at least one arginine-specific proteinase derived from P. gingivalis, preferably from Arg-gingipain, termed Arg-gingipain-1 (or RGP-1), and/or a Lys-gingipain, and/or a combination with another protein or other immunogen; or any other P. gingivalis virulence factor. Other immunogenic compositions or oligopeptides useful in the method of the invention are further described in U.S. Pat. No. 6,129,917, the entire contents of which are hereby incorporated by reference herein. The effectiveness of a candidate portion of P. gingivalis may be determined by testing such portion in the ApoE^{-/-} mouse model, as described herein.

Immunogenic compositions and/or vaccines may be formulated by any of the standard means known in the art. Such compositions are typically prepared as injectables, either as liquid solutions or suspensions. Solid forms suitable for solution in, or suspension in, liquid prior to injection may also be prepared. The preparation may also, for example, be emulsified, or the protein(s)/peptide(s) encapsulated in liposomes. Where mucosal immunity is desired, the immunogenic compositions advantageously contain an adjuvant such as the nontoxic cholera toxin B subunit (see, e.g., U.S. Pat. No. 5,462,734). Cholera toxin B subunit is commercially available, for example, from Sigma Chemical Company, St. Louis, Mo. While it is not necessary in the present invention, other suitable adjuvants are available and may be substituted therefor.

The active immunogenic ingredients are often mixed with excipients or carriers which are pharmaceutically acceptable and compatible with the active ingredient. Suitable excipients include but are not limited to water, saline, dextrose, glycerol, ethanol, or the like and combinations thereof. The concentration of the immunogenic polypeptide in injectable formulations is usually in the range of 0.2 to 5 mg/ml.

In addition, if desired, the vaccines may contain minor amounts of auxiliary substances such as wetting or emulsifying agents, pH buffering agents, and/or adjuvants which enhance the effectiveness of the vaccine. Examples of adjuvants which are effective include, but are not limited to, aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3hydroxyphosphoryloxy)-ethylamine (CGP 19835A, referred to as MTP-PE); and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogen resulting from administration of the immunogen in vaccines which also comprise the various adjuvants. Such additional formulations and modes of administration as are known in the art may also be used.

The immunogenic compositions or vaccines are administered in a manner compatible with the dosage formulation, and in such amount as are prophylactically and/or therapeutically effective. The quantity to be administered, generally in the range of about 100 to 1,000 μg of protein per dose, more generally in the range of about 5 to 500 μg of protein per dose, depends on the subject to be treated, the capacity of the individual's immune system to synthesize antibodies, and the degree of protection desired. Precise amounts of the immunogen may depend on the judgment of the medical professional and may be peculiar to each individual, but such a determination is within the skill of such a practitioner.

The vaccine or other immunogenic composition can be given in a single dose or multiple dose schedule. A multiple dose schedule is one in which a primary course of vaccination may include 1 to 10 or more separate doses, followed by other doses administered at subsequent time intervals as required to maintain and or reinforce the immune response, e.g., at 1 to 4 months for a second dose, and if needed, a subsequent dose(s) after several months.

The contents of all references, pending patent applications and published patent applications, cited throughout this application are hereby incorporated by reference.

### EXAMPLES

The following examples are presented to illustrate the advantages of the present invention and to assist one of ordinary skill in making and using the same. These examples are not intended in any way otherwise to limit the scope of the disclosure.

### Materials and Methods

Oral challenge and immunization. Porphyromonas gingivalis strain 381 and its fimbriae-deficient mutant DPG3 (FimA-³⁵) were grown as described³⁴. FimA- is impaired in attachment, and invasion of epithelial, and endothelial cells in vitro (Deshpande et al., 1998), fails to elicit inflammatory markers from these cells in vitro (Khlgatian et al., 2002; Nassar et al., 2002), and is not a potent stimulator of oral bone loss (Malek et al., 1994). Five-week-old male ApoE^{-/-} and C57BL-6 mice (Jackson Labs, Bar Harbor, Me.) were cared for in accordance with Boston University IACUC procedures, and received a standard chow diet. Mice were challenged with P. gingivalis 381 or FimA- in 2% carboxymethyl cellulose³⁶. Some groups of ApoE^{-/-} mice were immunized subcutaneously 2-times per week for 3 weeks with heat-killed P. gingivalis 381 whole organism preparations³⁶.

16S PCR of P. gingivalis and RT-PCR. One hundred μl of whole blood was collected from each mouse during the oral challenge regimen. Total DNA was collected using a QiaAmp Kit (Qiagen, Valencia, Calif.), and the P. gingivalis 16S gene was detected by PCR³⁷.

The aortic arch was harvested from ApoE^{-/-} mice following saline perfusion³⁸, and the tissue was homogenized with a sterile, RNAse-free tissue homogenizer. These samples were prepared for total RNA extraction using an RNEasy column (Qiagen), the fluid from the first column wash (DNA-enriched fraction) was collected, and used for 16S PCR of P. gingivalis. Total RNA was then collected per manufacturer's instructions and used for amplification of murine TLR-2, TLR-4, and β-actin.

Measurement of serum levels of P. gingivalis-specific IgG, cholesterol, and triglycerides. Serum levels of P. gingivalis-specific IgG was determined by ELISA. Total cholesterol (Sigma, St. Louis, Mo.), and triglycerides (Sigma) were determined per manufacturers instructions.

Assessment of periodontal bone loss. Oral bone loss was determined at the maxillary molars of all mice at 6-weeks of age as described previously³⁶.

Anti-mouse TLR-2 antibody. The TLR-2 monoclonal antibody was generated by immunizing Lewis rat with Chinese hamster ovary/mouse TLR-2 cells and fusing the spleenocytes to NSO/1 mouse myeloma cells. A rat IgG2b, k antibody clone was chosen based on recognition of Chinese hamster ovary/mouse TLR-2, and non-reactivity with Chinese hamster ovary/human TLR-2, or Chinese hamster ovary/human TLR-4 by FACS.

Assessment of Atherosclerosis and Immunohistochemistry. Animals were sacrificed 6-weeks after challenge, and were 17-weeks of age³⁹. The aorta of each mouse (n=10 mice per group) was harvested from the aortic valve to the iliac bifucation, opened longitudinally, and stained with Sudan IV^{29,39,40}. Digital micrographs were taken of the aortic arch, and the total area of atherosclerotic plaque was determined from onscreen images using IPLabs (Scanalytics, Inc., Fairfax, Va.) by an observer blinded to the identity of the samples. A subset of animals were perfused with saline, 4% paraformaldehyde, the aortic arch with heart tissue was harvested, and embedded. Eight-μm cryosections were collected, probed with anti-mouse TLR-2, anti-human TLR-4¹⁰, or isotype matched antibodies, developed, counterstained, and images recorded using a digital camera attached to a light microscope.

HAEC cell culture. Confluent monolayers of human aortic endothelial cells (HAEC; Cascade Biologics, Portland, Oreg.) in 6-well plates were challenged with wild type P. gingivalis or the FimA- mutant at a multiplicity of infection of 100. Similar cultures were incubated with either high (10 μg/ml) or low (1 μg/ml) doses of P. gingivalis FimA protein isolated from P. gingivalis 381⁴¹.

FACS. HAEC cultured for 2, 6 and 24 h with P. gingivalis wild type, the FimA- mutant, or FimA protein were washed, fixed, probed with FITC-labeled TLR-2, TLR-4 or isotype matched antibodies (Biocarta, San Diego, Calif.), and FACS analysis performed on 10,000 cells.

Statistical analysis. The data is presented as the mean±standard deviation. The student t test was employed to assess differences between groups, and a P<0.05 was considered significant.

### Example I

### Bacteremia and Localization of P. gingivalis in Aortic Arch Tissue of ApoE^{-/-} Mice Following Oral Infection

Experiments were performed to determine if oral infection of hyperlipidemic ApoE^{-/-} mice with wild type P. gingivalis strain 381 leads to dissemination of P. gingivalis (escape from the oral environment), as well as detection of P. gingivalis in the aortic tissues (localization of P. gingivalis in the tissues associated with accelerated atheroma formation). Groups of unchallenged C57BL-6 and ApoE^{-/-} mice were included to serve as age-matched control populations. Blood samples were obtained from ApoE^{-/-} mice 2 h after the final oral challenge with wild type or FimA- mutant, and the aortic arches were obtained 24 h after the final oral challenge. By PCR amplification of the P. gingivalis 16S rRNA gene, wild type P. gingivalis was detected in the blood of mice throughout the challenge regimen, while the FimA- mutant was only detected after the final oral challenge (Garcia et al., 1998). Additionally, PCR amplification of P. gingivalis 16S gene from ApoE^{-/-} mouse aortic arch tissue, at the site of predicted accelerated atheroma formation (Mach et al., 2002; Nakashima et al., 1994; Qiao et al., 1994; Tangirala et al., 1995) revealed that both wild type and FimA- mutant were present in these tissues.

### Example II

### Serum Analysis and Oral Bone Loss

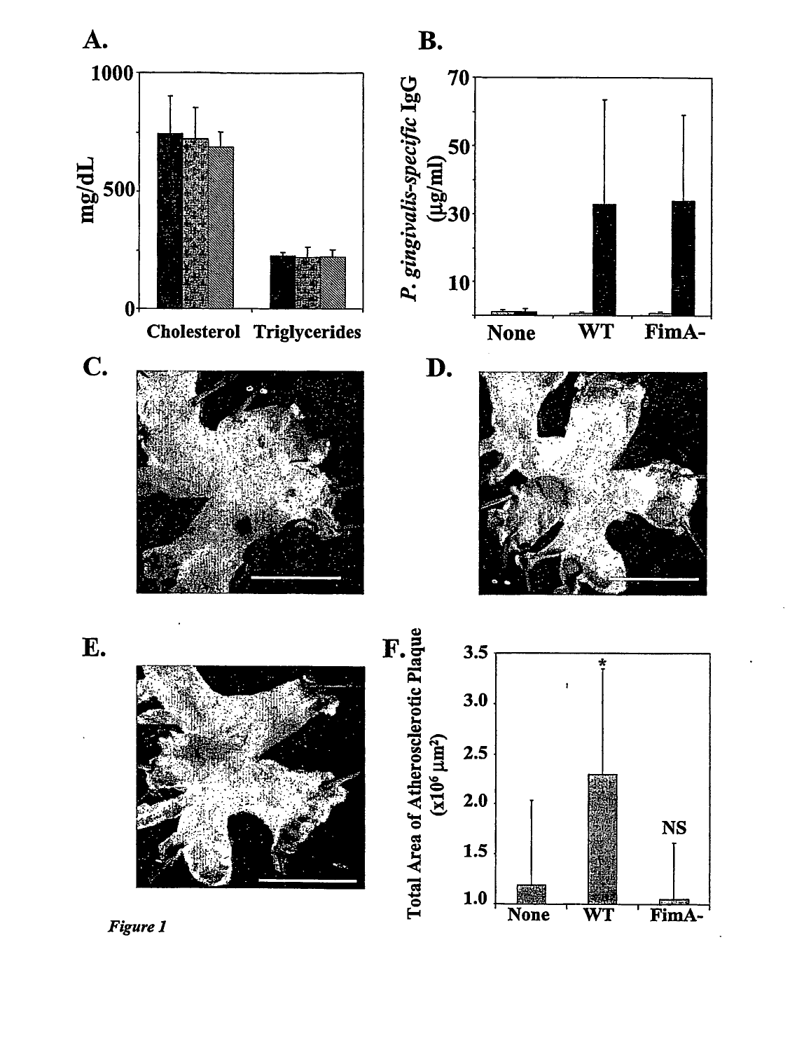

C57BL-6 mice, either unchallenged or orally challenged with either strain of P. gingivalis, possessed low cholesterol and triglyceride levels. Unchallenged ApoE^{-/-} mice possessed high cholesterol and triglyceride levels, and oral challenge with either the wild type or the P. gingivalis FimA- mutant had no effect on the serum levels of these molecules, demonstrating that P. gingivalis oral infection does not lead to modified serum lipid levels in the host (FIG. 1A). Analysis of sera for total P. gingivalis-specific IgG revealed no significant differences in the adaptive host response of ApoE^{-/-} mice following challenge with wild type P. gingivalis, or the FimA- mutant (FIG. 1B). C57BL/6 and ApoE^{-/-} mice developed oral bone loss to wild type P. gingivalis, while the FimA- mutant failed to stimulate oral bone loss (Malek et al., 1994).

### Example III

### P. gingivalis Oral Infection Accelerates Atherosclerosis in ApoE^{-/-} Mice

To determine if P. gingivalis oral infection leads to accelerated atheroma formation, the aorta of each mouse was harvested from the aortic valve to the iliac bifurcation and morphometric, and immunohistochemical analyses were performed as described above. It is well established that the accumulation of atherosclerotic plaque on the intimal surface of ApoE^{-/-} mouse aortas occurs at the aortic valves and continues into the aortic arch area (Mach et al., 2002; Qiao et al., 1994; Tangirala et al., 1995; Nakashima et al., 1994). Based on this, the total amount of atherosclerotic plaque present on the intimal surface from the aortic valves through the arch was determined. En face measurements of the Sudan IV stained atherosclerotic plaque accumulation revealed that ApoE^{-/-} mice challenged orally with wild type P. gingivalis possessed significantly more atherosclerotic plaque accumulation on the intimal surface of the aortic arch as compared to unchallenged ApoE^{-/-} mice (FIGS. 1C and 1D). However, despite detection of the P. gingivalis mutant in the blood 2 h after oral challenge, and in aortic arch tissue 24 h after oral challenge, ApoE^{-/-} mice challenged orally with the FimA- mutant failed to accelerate atheroma development, as the level of deposited atherosclerotic plaque resembled unchallenged animals (FIGS. 1E and 1F). Only fully invasive P. gingivalis initiated accelerated plaque accumulation, and we can infer that neither localization of P. gingivalis due to capture by the spontaneously developing plaque, nor the presence of P. gingivalis as demonstrated by PCR is sufficient to drive accelerated atheroma formation. The observed atherosclerotic plaque accumulation elicited by wild type P. gingivalis oral challenge did not progress to the thoracic or abdominal regions of the aorta of ApoE^{-/-} mice. The lack of progression of the plaque into these regions of the aorta may be a function of the chow diet, and age of these animals (17-weeks of age) (Nakashima et al., 1994; Lichtman et al., 1999).

### Example IV

### Invasive P. gingivalis Oral Infection Elicits TLR Expression in the Aortic Arch of ApoE^{-/-} Mice

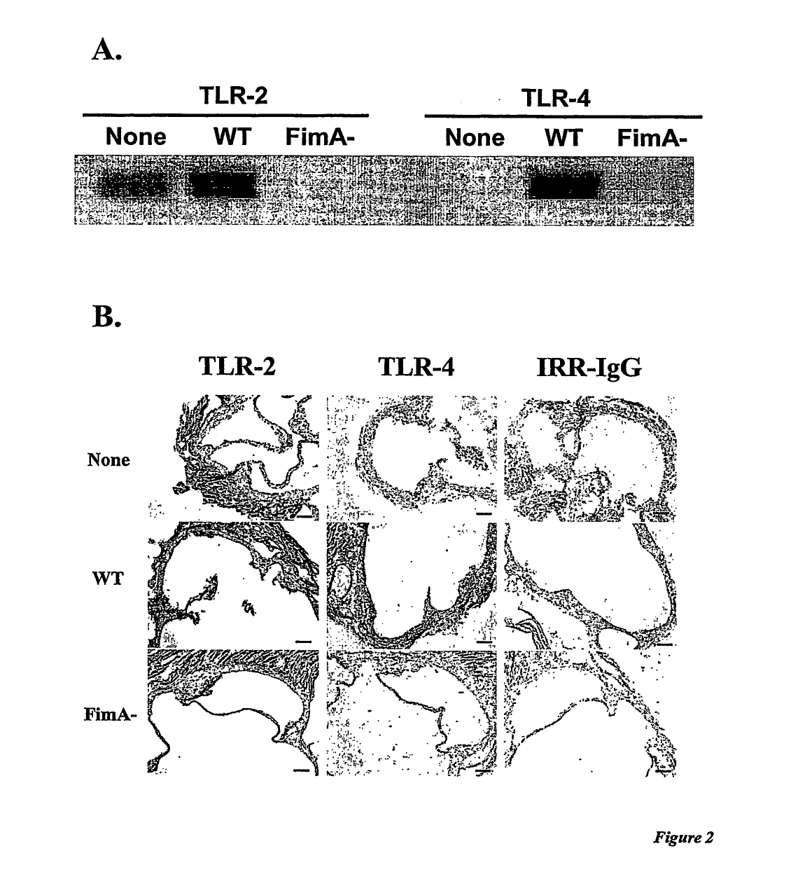

Since only the wild type P. gingivalis phenotype stimulated accelerated atheroma formation, and no differences in the adaptive immune response to wild type P. gingivalis or the mutant were observed, we next examined if differences in the innate immune response to P. gingivalis play a role in P. gingivalis-accelerated atheroma formation. Recently, it was reported that the pattern recognition receptor TLR-4, a marker of the innate immune response (Hansson et al., 2002), is up regulated in human, and mouse atheromatous vascular tissues (Xu et al., 2001). RT-PCR amplification for TLR-2, and TLR-4 in aortic tissue of P. gingivalis orally challenged ApoE^{-/-} mice revealed increased expression of TLR-2 and TLR-4 gene transcription in the mice orally challenged with wild type P. gingivalis. The P. gingivalis FimA- mutant challenged mice were negative for TLR-2 and TLR-4, and resembled unchallenged animals (FIG. 2A). TLR-2 and TLR-4 expression in the aortas of orally challenged ApoE^{-/-} mice was confirmed by immunohistochemistry (Xu et al., 2001; Nilsen et al., manuscript in preparation). TLR-2 was detected in aortic tissue of all ApoE^{-/-} mice and was localized primarily at atheromatous lesions. Low levels of TLR-2 were observed in aortic tissues of unchallenged mice (FIG. 2B). Elevated TLR-2 levels were observed in aortic tissue sections from ApoE^{-/-} mice orally challenged with wild type P. gingivalis. Slight TLR-2-specific staining was observed in tissue sections from mutant P. gingivalis challenged mice. TLR-4 expression was observed only in the aortic sinus of ApoE^{-/-} mice orally challenged with invasive P. gingivalis (FIG. 2B).

### Example V

### HAEC Infected with Wild Type P. gingivalis Express TLRs

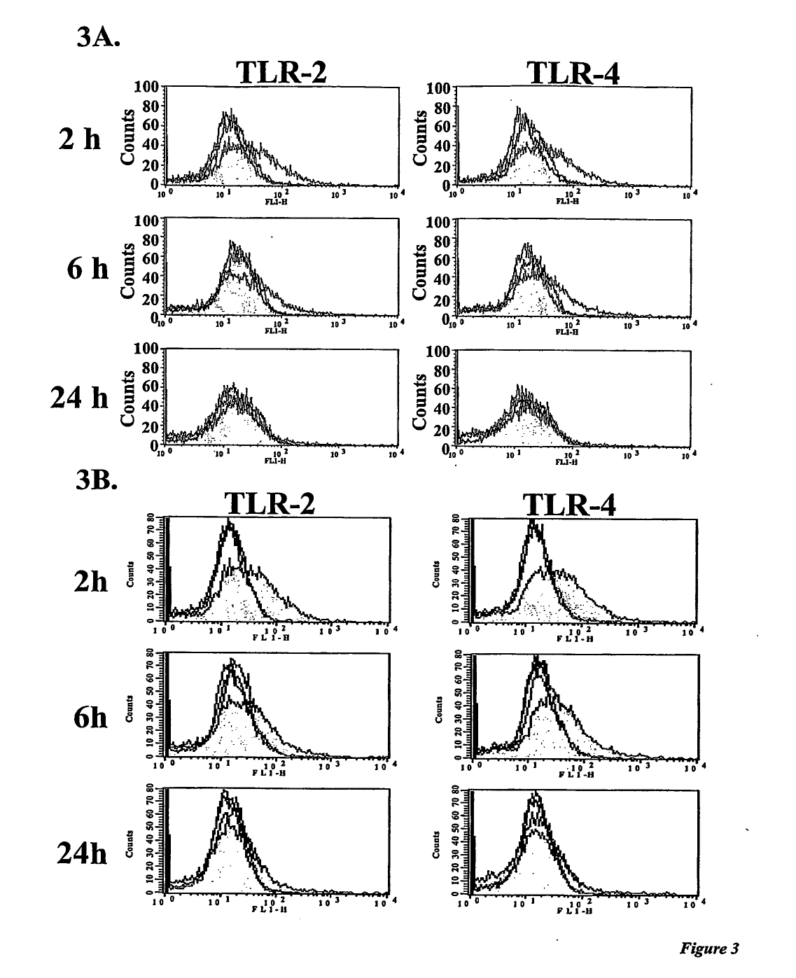

While aortic tissue from ApoE^{-/-} mice challenged with the P. gingivalis FimA- mutant failed to express TLR-4, and resembled unchallenged mice (FIG. 3B). FACS analysis revealed surface expression of TLR-2 and TLR-4 on HAEC cultured with wild type P. gingivalis at 2 and 6 h of co-culture. By 24 h post challenge TLR expression returned to levels similar to unstimulated cells. The FimA- mutant failed to stimulate TLRs, and resembled unstimulated cells (FIG. 3A). HAEC cultured with purified FimA protein did not express TLRs, and resembled unstimulated cells (FIG. 3B). These results suggest that P. gingivalis invasion, and not the FimA protein itself, was required for the up-regulation of TLR expression.

### Example VI

### Immunization to Prevent P. gingivalis-Elicited Periodontal Disease Ameliorates P. gingivalis-Accelerated Atherosclerosis in ApoE^{-/-} Mice

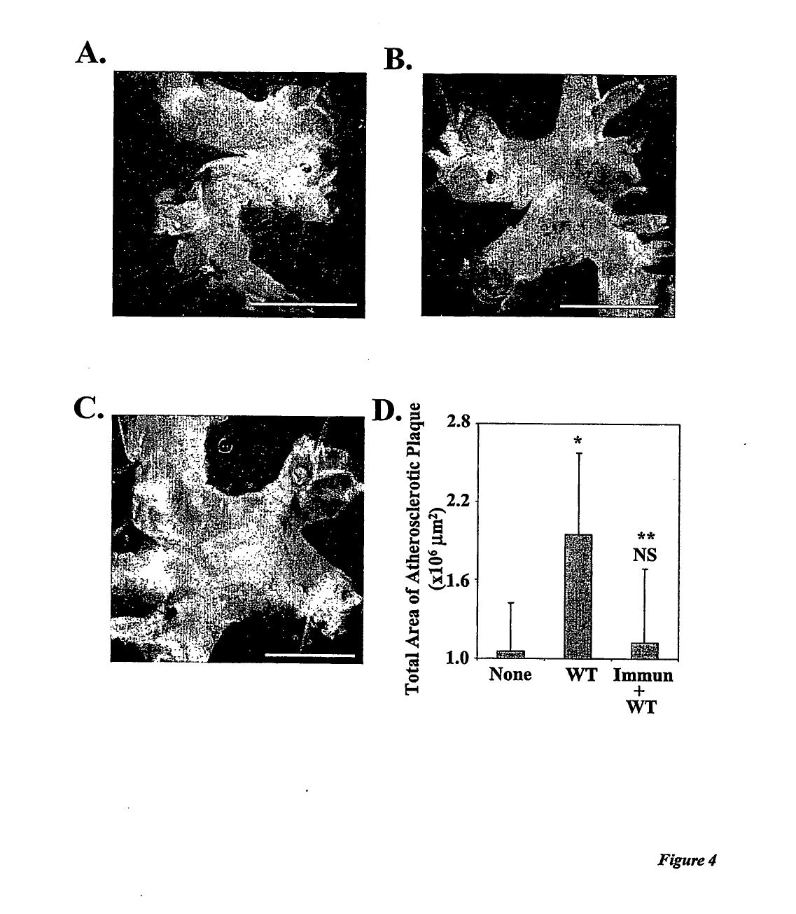

Immunization of mice with heat-killed P. gingivalis elicited potent P. gingivalis-specific IgG response, and prevented P. gingivalis-elicited oral bone loss (Gibson et al., 2001). Morphometric analysis of atherosclerotic plaque accumulation on the intimal surface of the aortic arch of mice revealed that immunization with heat-killed P. gingivalis protected animals from P. gingivalis-accelerated atherosclerotic plaque accumulation (FIGS. 4A-D). These results demonstrate that immunization prevents both P. gingivalis-mediated periodontal disease, and P. gingivalis-accelerated atherosclerosis.

### REFERENCES

Armitage (1996) “Periodontal diseases: diagnosis.” Ann. Periodontol. 1:37-215;
Asai et al. (2001) “Bacterial fimbriae and their peptides activate human gingival epithelial cells through Toll-like receptor 2.” Infect. Immun. 69:7387-95;
Bainbridge et al. (2001) “Porphyromonas gingivalis lipopolysaccharide: an unusual pattern recognition receptor ligand for the innate host defense system.” Acta Odontol. Scand. 59:131-8;
Beck et al. (1998) “Periodontitis: a risk factor for coronary heart disease?” Ann. Periodontol. 3:127-41;
Beck et al. (2001) “Relationship of periodontal disease to carotid artery intima-media wall thickness: the atherosclerosis risk in communities (ARIC) study.” Arterioscler. Thromb. Vasc. Biol. 21:1816-22;
Cybulsky et al., (2001) “A major role for VCAM-1, but not ICAM-1, in early atherosclerosis.” J. Clin. Invest. 107:1255-62;
Deshpande et al. (1998) “Invasion of aortic and heart endothelial cells by Porphyromonas gingivalis.” Infect. Immun. 66:5337-43;
Deshpande et al. (1998) Invasion Metastasis, 18:57-59;
Epstein et al. (1999) “Potential role of cytomegalovirus in the pathogenesis of restenosis and atherosclerosis.” Am. Heart J. 138:S476-8;
Epstein et al. (1999) Circulation 100:e20-8;
Garcia et al. (1998) “Rapid detection of Actinobacillus actinomycetemcomitans, Prevotella intermedia and Porphyromonas gingivalis by multiplex PCR.” J. Periodontal Res. 33:59-64;
Griffen et al. (1998) “Prevalence of Porphyromonas gingivalis and periodontal health status.” J. Clin. Microbiol. 36:3239-42;
Gupta et al. (1997) “IFN-gamma potentiates atherosclerosis in ApoE knock-out mice.” J. Clin. Invest. 99:2752-61;
Hajishengallis et al. (2002) “Dependence of bacterial protein adhesins on toll-like receptors for proinflammatory cytokine induction.” Clin. Diagn. Lab. Immunol. 9:403-11;
Hansson et al. (2002) “Innate and adaptive immunity in the pathogenesis of atherosclerosis.” Circ. Res. 91:281-91;
Haraszthy et al. (2000) “Identification of periodontal pathogens in atheromatous plaques.” J. Periodontol. 71:1554-60;
Holt et al. (1988) “Implantation of Bacteroides gingivalis in nonhuman primates initiates progression of periodontitis.” Science. 239:55-7;
Holt et al. (1999) “Virulence factors of Porphyromonas gingivalis.” Periodontol 2000 20:168-238;
Hujoel et al. (2000) “Periodontal disease and coronary heart disease risk.” Jama. 284:1406-10;
Khlgatian et al. (2002) “Fimbria-dependent activation of cell adhesion molecule expression in Porphyromonas gingivalis-infected endothelial cells.” Infect. Immun. 70:257-67;
Libby (2001) “What have we learned about the biology of atherosclerosis? The role of inflammation.” Am. J. Cardiol. 88:3J-6J;
Lien et al. (2002) “Toll-like receptors.” Crit. Care Med. 30:S1-11;
Liuzzo et al. (1994) “The prognostic value of C-reactive protein and serum amyloid a protein in severe unstable angina.” N. Engl. J. Med., 331:417-24;
Mach et al. (1999) “Differential expression of three T lymphocyte-11 activating CXC chemokines by human atheroma-associated cells.” J. Clin. Invest. 104:1041-50;
Mach et al. (2002) “Influence of Helicobacter pylori infection during atherogenesis in vivo in mice.” Circ. Res. 90:E1-4;
Malek et al. (1994) “Inactivation of the Porphyromonas gingivalis fimA gene blocks periodontal damage in gnotobiotic rats.” J. Bacteriol. 176:1052-9;
Nakashima et al. (1994) “ApoE-deficient mice develop lesions of all phases of atherosclerosis throughout the arterial tree.” Arterioscler. Thromb. 14:133-240;
Nassar et al. (2002) “Role for fimbriae and lysine-specific cysteine proteinase gingipain K in expression of interleukin-8 and monocyte chemoattractant protein in Porphyromonas gingivalis-infected endothelial cells.” Infect. Immun. 70:268-76;
Oliver et al. (1998) “Periodontal diseases in the United States population.” Periodontol. 69:269-78;
Plump et al. (1992) “Severe hypercholesterolemia and atherosclerosis in apolipoprotein E-deficient mice created by homologous recombination in ES cells.” Cell. 71:343-53;
Qiao et al. (1994) “Pathology of atheromatous lesions in inbred and genetically engineered mice. Genetic determination of arterial calcification.” Arterioscler. Thromb. 14:1480-97;
Ross (1993) “The pathogenesis of atherosclerosis: a perspective for the 1990s.” Nature 362:801-9;
Shuto et al. (2002) “Glucocorticoids synergistically enhance nontypeable Haemophilus influenzae-induced Toll-like receptor 2 expression via a negative cross-talk with p38 MAP kinase.” J. Biol. Chem. 277:17263-70;
Steinberg (1997) “Low density lipoprotein oxidation and its pathobiological significance.” J. Biol. Chem. 272:20963-6;
Tangirala et al. (1995) “Quantitation of atherosclerosis in murine models: correlation between lesions in the aortic origin and in the entire aorta, and differences in the extent of lesions between sexes in LDL receptor-deficient and apolipoprotein E-deficient mice.” J. Lipid Res. 36:2320-8;
Wang et al. (2000) “Regulation of toll-like receptor 2 expression by macrophages following Mycobacterium avium infection.” J. Immunol. 165:6308-13;
Wright et al. (2000) “Infectious agents are not necessary for murine atherogenesis.” J. Exp. Med. 191:1437-42;
Wu et al. (2000) “Periodontal disease and risk of cerebrovascular disease: the first national health and nutrition examination survey and its follow-up study.” Arch. Intern. Med. 160:2749-55;
Xu et al. (2001) “Toll-like receptor-4 is expressed by macrophages in murine and human lipid-rich atherosclerotic plaques and upregulated by oxidized LDL.” Circulation 104:3103-8; and
Zhang et al. (1992) “Spontaneous hypercholesterolemia and arterial lesions in mice lacking apolipoprotein E.” Science. 258:468-71.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A method of therapeutically treating a patient having a symptom of cardiovascular disease, said method comprising the steps of:
(a) providing a patient having a symptom of cardiovascular disease; and
(b) administering to said patient a therapeutic amount of an immunogenic composition comprising an immunogenically effective portion of Porphyromonas gingivalis in a pharmaceutically effective carrier substance.

2. A method of prophylactically treating a patient having a risk factor for cardiovascular disease, said method comprising the steps of:
(a) providing a patient having a risk factor for cardiovascular disease; and
(b) administering to said patient a prophylactic amount of an immunogenic composition comprising an immunogenically effective portion of Porphyromonas gingivalis in a pharmaceutically effective carrier substance.

3. The method of claim 1 or claim 2, wherein said immunogenically effective portion of Porphyromonas gingivalis is selected from the group consisting of P. gingivalis heat-killed whole cells; an immunogenically effective portion of P. gingivalis capsular polysaccharide; and an immunogenic composition comprising at least one peptide corresponding in sequence to the amino terminus of at least one arginine-specific proteinase derived from P. gingivalis.

4. The method of claim 1, wherein said symptom of cardiovascular disease is an elevated level of C-reactive protein.

5. The method of claim 1, wherein said symptom of cardiovascular disease is atherosclerosis in said patient.

6. The method of claim 1, wherein said patient has atheroma formation in the aortic arch.

7. The method of claim 2, wherein said patient furthermore shows a symptom of cardiovascular disease.

8. A vaccine against cardiovascular disease comprising a therapeutically effective quantity of an immunogenically effective portion of heat-killed Porphyromonas gingivalis.

9. The vaccine of claim 8, wherein said immunogenically effective portion of heat-killed Porphyromonas gingivalis is heat-killed whole cells.

10. The vaccine of claim 8, said vaccine being effective in preventing or treating atheroma formation.

11. The vaccine of claim 8, said vaccine being effective in preventing or treating atherosclerosis.

12. The vaccine of claim 8, said vaccine further comprising a pharmaceutically acceptable carrier.
